(19) United States
(12) Patent Application Publication
(10) Pub. No.: US 2007/0184436 A1
(43) Pub. Date: Aug. 9, 2007

(54) Generic capture probe arrays

(76) Inventors: Joel Myerson, Berkeley, CA (US); Jeffrey R. Sampson, Burlingame, CA (US)

Correspondence Address: AGILENT TECHNOLOGIES, INC.;Legal Department, DL429, Intellectual Property Administration, P. O. Box 7599, Loveland, CO, 80537-0599, US

(21) Appl. No.: 09/876,589

(22) Filed: Jun. 7, 2001

(51) Int. Cl. C12Q 1/68; C12M 3/00

(52) U.S. Cl. 435/6; 435/287.2

(57) ABSTRACT

The invention provides generic capture probe arrays, methods of making generic capture probe arrays, and methods of using these arrays to detect target analytes in samples.

This invention relates to arrays of capture probes that can be used in diagnostic and analytical methods.

### BACKGROUND OF THE INVENTION

Detection of specific interactions between biological molecules, such as nucleic acid molecules, is employed in a wide variety of research, medical, and industrial applications, including the detection of disease-related molecules in diagnostic assays, screening for clones of novel target polynucleotides, mapping of genetic loci, nucleic acid molecule sequencing, and detection of pollutants in environmental samples.

Analysis of specific binding interactions can take place in numerous formats. Of particular interest are formats that allow the processing of a large number of samples at a time, such as probe arrays. There are generally two approaches to creating large arrays of probes, such as oligonucleotide probes. Either the probes are synthesized on a surface in a correct spatial array (“in situ synthesis”), or pre-made probe oligomers are synthesized off-line and bound to correct locations on a surface as whole oligomers (“whole oligo deposition”). These techniques each have advantages and disadvantages.

In situ synthesis is well-suited for making small numbers of arrays of oligonucleotide probes. Arrays made using this method are generally made one at a time, making it easy to make changes in the composition of a single array. However, there is a significant chemical challenge to have DNA synthesis chemistry, which is extremely sensitive to small amounts of water, work consistently and reliably with picoliter deliveries of reagents, as is required for in situ synthesis. This challenge is more pronounced when using standard DNA synthesis chemistry, in which an oxidation step requires the addition of a large quantity of water. The difficulty of quality control of the manufactured arrays is significant. Although this problem can be minimized by judicious design, because each array is synthesized separately, rather than by a batch process, there is no way to ensure that each array is fully functional without testing it. Also, this process is time-consuming and inefficient, particularly when a large number of arrays are desired.

Whole oligonucleotide deposition is well-suited for making large numbers of identical arrays. Synthesis of oligonucleotides off-line allows the use of more robust synthesis techniques, on a larger scale, as well as separate quality control of individual oligonucleotides. A challenge presented in the use of such methods is in efficiently transferring individual oligonucleotides to proper locations on a solid support to generate an array. Many methods have been developed in efforts to accomplish this, none of which are particularly rapid, inexpensive, or easy.

### SUMMARY OF THE INVENTION

The invention provides generic nucleic acid molecule capture probe arrays, methods for making such arrays, and methods of using such arrays, for example, in diagnostic and analytical applications. Production of the arrays of the invention is rapid and efficient, and the arrays of the invention can be adapted for use in innumerable applications.

In particular, the arrays include capture probes, which are bound to a solid support, and solution probes, which each contain (i) a first region that binds to an immobilized capture probe (the “α-capture” or “αC” region), and (ii) a second region that binds to a target analyte (the “α-target” or “αT” region). A single array of capture probes, thus, can be adapted for use in many applications, by changing the specificity of the α-target regions of solution probes, rather than by changing the array of capture probes.

Central to the arrays of the invention is covalent linkage of solution probes to a solid support. This linkage provides significant advantages to the arrays of the invention. For example, this interaction enables the arrays to be used in assays carried out under high stringency conditions, which could result in separation of capture and solution probes in the absence of such linkage. Use of such assay conditions may be required, for example, in detecting highly specific interactions, e.g., in distinguishing the binding properties of closely related species. The linkage also enables the use of relatively short regions of binding between capture and solution probes, as long regions are not required to provide strength to the binding of a solution probe to the solid support. Rather, strength is provided by the direct linkage of the solution probe to the solid support. Thus, the arrays of the invention provide all of the benefits of using arrays on which the probes are unimolecular (e.g., the lack of possibility of bimolecular probes separating from one another under highly stringent assay conditions), without encountering problems of such arrays, such as the need to synthesize a new array for each application. Also, the arrays of the invention maintain the cost effectiveness and synthetic simplicity of arrays on which the probes are bimolecular, e.g., the ability to use a single array of capture probes for numerous applications.

Accordingly, in a first aspect, the invention provides an array of probes linked to a solid support. This array includes a plurality of (a) nucleic acid molecule capture probes bound to the solid support, and (b) nucleic acid molecule solution probes that each include (i) a first region that is specifically bound to a capture probe, and (ii) a second region that specifically binds to a target nucleic acid molecule analyte. At least some of the solution probes are covalently linked directly to the solid support by a chemical moiety attached to the solution probes that, in an activated state, covalently binds to the support. In addition, the interaction between at least some of the solution probes and at least some of the capture probes can, optionally, involve interaction between nonstandard or reverse polarity bases or covalent crosslinking. The array can include different solution probes that are specific for distinct target nucleic acid molecule analytes.

In a second aspect, the invention provides a method of making an array of nucleic acid molecule probes for detecting a target nucleic acid molecule analyte in a sample. This method involves (a) providing an array including nucleic acid molecule capture probes bound to a solid support; (b) contacting the array of step (a) with nucleic acid molecule solution probes that each include (i) a first region that specifically binds to a capture probe, and (ii) a second region that specifically binds to a target nucleic acid molecule analyte; and (c) covalently linking at least some of the solution probes to the solid support by way of an activatable chemical moiety attached to the solution probes. The interaction between at least some of the solution probes and at least some of the capture probes can, optionally, involve interaction between nonstandard or reverse polarity bases, or covalent crosslinking. The array used in this method can include capture probes that are specific for different solution probes, which are specific for distinct target nucleic acid molecule analytes.

In a third aspect, the invention provides a method of detecting a target nucleic acid molecule analyte in a sample. This method involves (a) contacting a nucleic acid molecule capture probe, bound to a solid support, with a nucleic acid molecule solution probe including (i) a first region that specifically binds to a target analyte, and (ii) a second region that specifically binds to the capture probe; (b) covalently linking the solution probe to the solid support; (c) contacting the product of (b) with the sample; and (d) monitoring the solid support for the presence of the target nucleic acid molecule analyte bound to the capture probe. This method can, optionally, further include covalently crosslinking at least some of the solution probes to at least some of the capture probes. Also, the method can be used to detect a second (or more) target nucleic acid molecule analyte in a sample, or can be used to detect a particular target nucleic acid molecule analyte in more than one sample.

In a fourth aspect, the invention provides a method of detecting a target nucleic acid molecule analyte(s) in a sample(s), which is identical to that described in the third aspect, except that the solution probe and the sample are contacted with one another prior to their contact with the capture probe.

Molecules, such as nucleic acid molecules, are stated herein to “specifically bind” to one another if they, in their relationship to one another (e.g., as hybridizing oligonucleotides) bind to one another with greater affinity than to unrelated molecules, such as unrelated molecules that may be present in a sample or reaction mixture containing the specifically binding molecules. For example, nucleic acid molecules can be said to “specifically bind” to one another in the invention if they hybridize to one another under at least low stringency conditions, but, preferably, under high stringency conditions.

An example of high stringency conditions includes hybridization at about 42° C. in about 50% formamide, 0.1 mg/ml sheared salmon sperm DNA, 1% SDS, 2×SSC, 10% dextran sulfate, a first wash at about 65° C., about 2×SSC, 1% SDS, followed by a second wash at about 65° C. and in about 0.1×SSC. Alternatively, high stringency conditions can include hybridization at about 42° C. and in about 50% formamide, 0.1 mg/ml sheared salmon sperm DNA, 0.5% SDS, 5×SSPE, 1× Denhardt's, followed by two washes at room temperature and in 2×SSC, 0.1% SDS, and two washes at about 55-60° C. and in 0.2×SSC, 0.1% SDS.

An example of low stringency hybridization conditions includes hybridization at about 42° C. and in 0.1 mg/ml sheared salmon sperm DNA, 1% SDS, 2×SSC, and 10% dextran sulfate (in the absence of formamide), and a wash at about 37° C. and in 6×SSC, 1% SDS. Alternatively, a low stringency hybridization can be carried out at about 42° C. and in 40% formamide, 0.1 mg/ml sheared salmon sperm DNA, 0.5% SDS, 5×SSPE, 1× Denhardt's, followed by two washes at room temperature and in 2×SSC, 0.1% SDS, and two washes at room temperature and in 0.5×SSC, 0.1% SDS. These stringency conditions are exemplary; other appropriate conditions may be determined by one of skill in this art.

The arrays of the invention can be used in innumerable applications that are known to those of skill in this art. For example, as is described further below, the arrays can be used to detect target nucleic acid molecule analytes in samples, for example, in medical diagnostic methods and other analytical methods. In addition to these methods, the arrays of the invention can be used, for example, for genetic mapping (Khrapko et al., DNA Seq. 1(6):375-388, 1991), genetic identification, nucleic acid sequencing (e.g., multiplex DNA sequencing; Church et al., Science 240:185-188, 1998), DNA and RNA fingerprinting, construction and use of combinatorial chemical libraries, and tracking, retrieving, and identifying compounds labeled with oligonucleotide tags.

The invention provides several advantages. For example, a single capture probe array can be used to generate innumerable different arrays by the use of different solution probes. This eliminates time-consuming, expensive, and technically difficult customization of capture probe arrays for every single application. Also, binding between target analytes and solution probes can be carried out in solution, in the absence of a solid support (i.e., a capture probe array), which can be more effective with certain molecules (e.g., oligonucleotide molecules having secondary structure). In some embodiments of the invention, oligonucleotide probes are synthesized and then attached to a solid support. Such off-line synthesis allows quality control analysis of the oligonucleotides before they are applied to a solid support. Also, the invention requires increasing the stability of interactions between solution probes and a solid support, e.g., by covalent linkage, enabling higher stringency reaction conditions to be used, thus eliminating non-specific binding. Spatial information is not destroyed with such arrays, if the temperature of the system is heated above the melting temperature of hybridizing regions of probes.

Other features and advantages of the invention will be apparent from the following detailed description, the drawings, and the claims.

### BRIEF DESCRIPTION OF THE DRAWINGS

FIG. 1 is a schematic illustration of an array of the invention, showing capture probes (C1, C2, and Cn) bound to a solid support, at Features 1, 2, and n, respectively, to form a capture surface.

FIG. 2 is a schematic illustration of a method of the invention, in which a pool of target molecules (T1, T2, and Tn) and a pool solution probes (αT1-αC1, αT2-αC2, and αTn-αCn) are pre-incubated with one another prior to contact with a capture surface.

FIG. 3 is a schematic illustration of an array of the invention, to the capture probes (C1, C2, and C3) of which are bound solution probes (αT1-αC1, αT2-αC2, and αTn-αCn) and target molecules (T1, T2, and Tn). As is discussed below, the solution probes are covalently linked to the solid support (and optionally, to capture probes, or both).

FIG. 4 is a schematic illustration of a method of the invention, in which solution probes (αT1-αC1, αT2-αC2, and αTn-αCn) and capture probes (C1, C2, and C3), immobilized on a capture surface, are pre-incubated with one another prior to contact with target molecules. As is discussed below, the solution probes are covalently linked to the solid support (and optionally, to capture probes, or both).

FIG. 5 is a schematic illustration of a method of the invention, in which target mRNAs (mRNA-1, mRNA-2, and mRNA-n) and solution probes including α-target regions (cDNA-1 or PCR product-1, cDNA-2 or PCR product-2, and cDNA-n or PCR product-n) are pre-incubated with one another prior to contact with a capture surface.

FIG. 6 is a schematic illustration of an array of the invention, to the capture probes (C1, C2, and Cn) of which are bound solution probes, via α-capture probe regions (αC1, αC2, and αCn), that are also bound to target molecules (mRNA-1, mRNA-2, and mRNA-n), via α-target regions (cDNA-1 or PCR product-1, cDNA-2 or PCR product-2, and cDNA-n or PCR product-n). As is discussed below, the solution probes are covalently linked to the solid support (and optionally, to capture probes, or both).

FIG. 7 is a schematic illustration of a method of the invention, in which modified bases (X and W) are used to prevent undesired hybridization of target molecules to a solution probe.

FIG. 8 is a schematic illustration of a method of the invention, in which covalent linkage of a solution probe to a capture probe, via bases Y and Z, is used to prevent non-specific binding to a capture probe.

### DETAILED DESCRIPTION

Detection of specific interactions between biological molecules, such as nucleic acid molecules, is fundamental to numerous diagnostic and analytical applications in biology and medicine. Many of these applications have been facilitated by the use of arrays of specific probe molecules that are immobilized on solid supports. Manufacture of such arrays can be difficult, time-consuming, and expensive, which can hinder their use in a wide variety of applications.

As is noted above, the invention provides probe arrays that, rather than being limited to a particular use, can be adapted for use in innumerable applications. In particular, the probe arrays of the present invention include capture probes, which are bound to a solid support, and solution probes, which each contain (i) a first region that binds to an immobilized capture probe (the “α-capture” or “αC” region), and (ii) a second region that binds to a target analyte (the “α-target” or “αT” region). As is discussed further below, interaction between the capture and solution probes can involve basepairing between standard or modified nucleotides. This interaction can also be strengthened by interstrand crosslinking. In any case, according to the invention, a single array of capture probes can be adapted for use in many applications, by changing the specificity of the α-target regions of solution probes, rather than by changing the array of capture probes.

A central feature of the probe arrays of the invention is that at least some of the solution probes are bound directly to the solid support. The basepairing interaction between the solution and capture probes provides specificity as to where the solution probes are localized on the support, as well as some strength to the interaction of the solution probes to the support, however, the solution probes are also linked directly to the surface, for example, by use of a linker molecule. This central feature of the invention is described further below.

In addition to the arrays described above (also see below), the invention includes methods of making these arrays and methods of their use in, for example, detecting target analytes in samples. The arrays and methods of the invention are described in further detail, as follows.

Capture Probe Arrays

As is discussed above, a fundamental feature of the arrays and methods of the invention is a generic nucleic acid molecule capture probe array, which can be adapted, by the use of solution probes, for use in many applications. Each capture probe in the generic capture arrays consists of a region that binds to a solution probe (in particular, to the αC region of a solution probe), as well as a region that links the capture probe to a solid support. The capture probes of the invention oligonucleotides contain DNA, RNA, or modifications thereof.

Methods for synthesizing or obtaining oligonucleotide probe molecules are well known in the art. For example, such probes can be made using an automated DNA synthesizer, e.g., an Applied Biosystems, Inc. (Foster City, Calif.) model 392 or 394 DNA/RNA Synthesizer, and standard chemical methods, such as phosphoramidite chemistry, which can be adapted as needed for incorporation of modified or nonstandard bases, if desired (see, e.g., Beaucage et al., Tetrahedron 48:2223-2311, 1992; Molko et al., U.S. Pat. No. 4,980,460; Koster et al., U.S. Pat. No. 4,725,677; Caruthers et al., U.S. Pat. Nos. 4,415,732, 4,458,066, and 4,973,679). Alternative chemistries that result in non-natural backbone groups, such as phosphorothioate, methylphosphonate, or phosphoramidate backbones, can also be employed to make oligonucleotide capture probes of the invention, provided that the resulting oligonucleotides are capable of specific hybridization. In some embodiments of the invention, oligonucleotides can include nucleotides that permit processing or manipulation by enzymes, or non-naturally occurring nucleotide analogs, such as peptide nucleic acids, that promote the formation of more stable duplexes than standard nucleotides.

The portion of the capture probe that specifically binds to a solution probe (and thus the corresponding region of the solution probe) can range in length, for example, from 6-60 nucleotides, e.g., 12-40 nucleotides or 28-35 nucleotides, and specifically hybridizes to the αC region of the solution probe by Watson-Crick base pairing. Of course, one of skill in this art can vary the lengths of such interacting regions, depending on specific parameters of an assay, such as the temperature, the base content of hybridizing regions (A/T vs. G/C content), the presence of modified bases (see below), the use of agents to crosslink hybridizing regions (also see below), and the use of reagents that negate base-specific stability differences of duplexes (e.g., tetramethylammonium chloride).

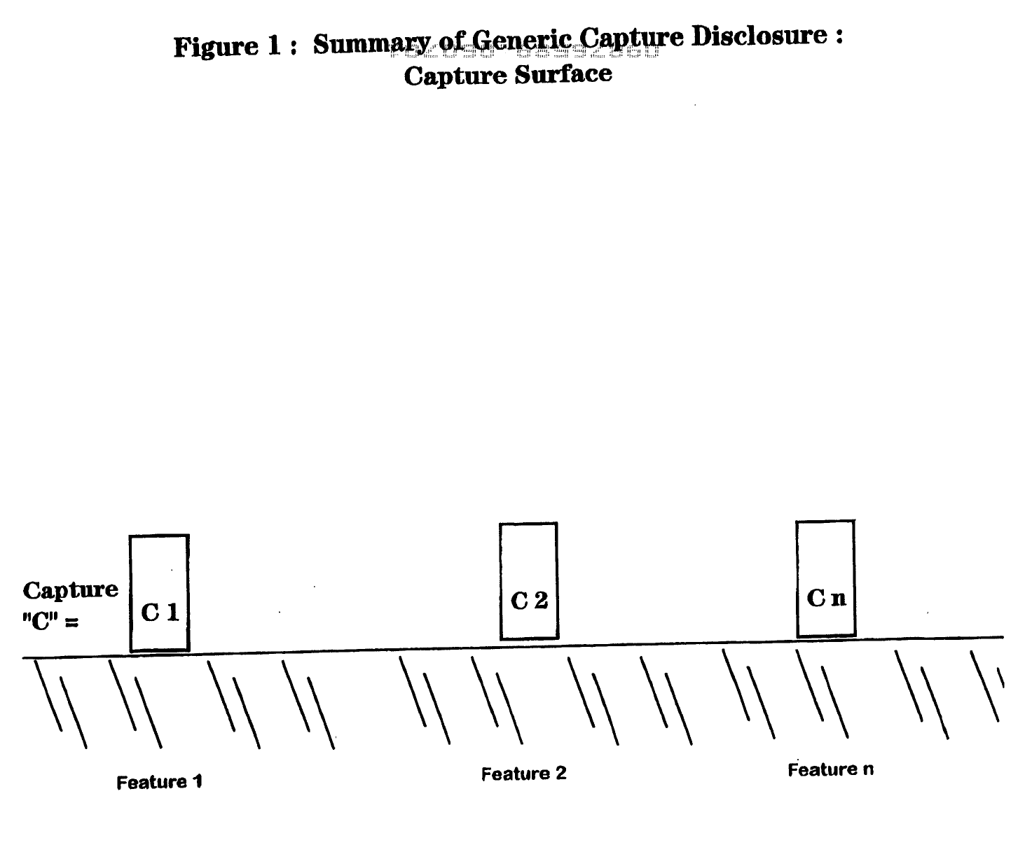

Different capture probes to be included in a capture array are bound to a solid support in discrete, predetermined areas (referred to herein as “features”; FIG. 1). The number of probes bound to each feature will vary, depending on the type of capture probe used and the specific application, and can readily be determined by one of skill in this art. For example, an individual feature of an array, which includes identical probes, may include more than 10,000 probes/μm². Also, the size of each feature can vary according to the particular use, and can range, for example, from several μm², e.g., 10-20, to several thousand μm², e.g., 1000-30,000 μm². Preferably, the features are spatially discrete, so that signals generated by events, such as fluorescent emissions, at adjacent features can be resolved by use of a standard detection method.

Capture probe arrays are fabricated on solid supports, such as, for example, glass (e.g., glass microscope slides or coverslips), plastic, alkanethiolate-derivatized gold, cellulose, polystyrene, silica gel, polyamide, functionalized glass, Si, Ge, GaAs, GaP, SiO₂, SIN₄, modified silicone, polymerized Langmuir Blodgett film, or any one of a wide variety of polymers, such as (poly)tetrafluoroethylene, (poly)vinylidenedifluoride, or combinations thereof. For example, the solid support can be a flat glass or single-crystal silicon with surface features of less than 10 angstroms.

The solid support can be coated with a surface material, such as a polymer, plastic, resin, polysaccharide, silica, silica-based material, carbon, metal, inorganic glass, or membrane, as can be selected by one of skill in this art. It may be desirable for the surface of the solid support to include a layer of crosslinking groups. For example, when thiols are used to link probes to the surface, solid supports coated with an intermediate linker layer such as aryl acetylenes, ethylene glycol oligomers, diamines, diacids, amino acids, or combinations thereof, can be used (see, e.g., U.S. Pat. No. 5,412,087).

The capture probes can be synthesized directly on a feature of a solid support or synthesized elsewhere, and then added as an intact species that is covalently linked to the feature of the substrate. Numerous methods (e.g., photolithographic methods; see, e.g., Sze, VLSI Technology, McGraw-Hill, 1983; Mead et al., Introduction to VLSI Systems, Addison-Wesley, 1980) for attaching biological polymers, such as oligonucleotides (DNA or RNA), proteins, peptides, and carbohydrates, to solid supports are known in the art, and can be used to make the capture arrays of the invention. For example, McGall et al. (U.S. Pat. No. 5,412,087) describes a process in which a substrate is coated with compounds having thiol functional groups that are protected with photoremovable protecting groups. Probes, such as oligonucleotide probes or other biological polymers, can be linked to different regions of such a substrate by spatial irradiation, which results in removal of protecting groups at pre-defined regions of the surface.

Other methods for attaching probe molecules to solid supports that can be used to make the capture arrays of the invention are described, for example, in U.S. Pat. No. 4,681,870, which describes a method for introducing free amino or carboxyl groups onto a silica matrix; the carboxyl groups can be subsequently covalently linked to a polypeptide in the presence of carbodimide. U.S. Pat. No. 4,762,881 describes a method for attaching a polypeptide to a solid support by incorporating a light-sensitive, unnatural amino acid group into the polypeptide chain, and exposing the product to low-energy ultraviolet light.

Additional methods for attaching molecules, such as oligonucleotides, onto solid supports are described, for example, in U.S. Pat. No. 5,601,980, U.S. Pat. No. 4,542,102, WO 90/07582, U.S. Pat. No. 4,937,188, U.S. Pat. No. 5,011,770, WO 91/00868, U.S. Pat. No. 5,436,327, U.S. Pat. No. 5,143,854, WO 90/15070, Fodor et al., Science 251:767-773, 1991, Dower et al., Ann. Rev. Med. Chem. 26:271-280, 1991, U.S. Pat. No. 5,252,743, WO 91/07087, U.S. Pat. No. 5,445,934, U.S. Pat. No. 5,744,305, and U.S. Pat. No. 5,624,711. Also see U.S. Pat. Nos. 5,604,097, 5,635,400, 5,654,413, and 5,695,934.

Solution Probes

As is noted above, each solution probe consists of a first region that specifically binds to a capture probe (the αC region) and a second region that specifically binds to a target analyte (the αT region). These two regions of a solution probe typically are covalently linked to one another, for example, they may be part of a single oligonucleotide or crosslinked to one another, but may be linked by other means as well, as is known in the art. Solution probes can be made using standard methods, such as those described above in reference to capture probes.

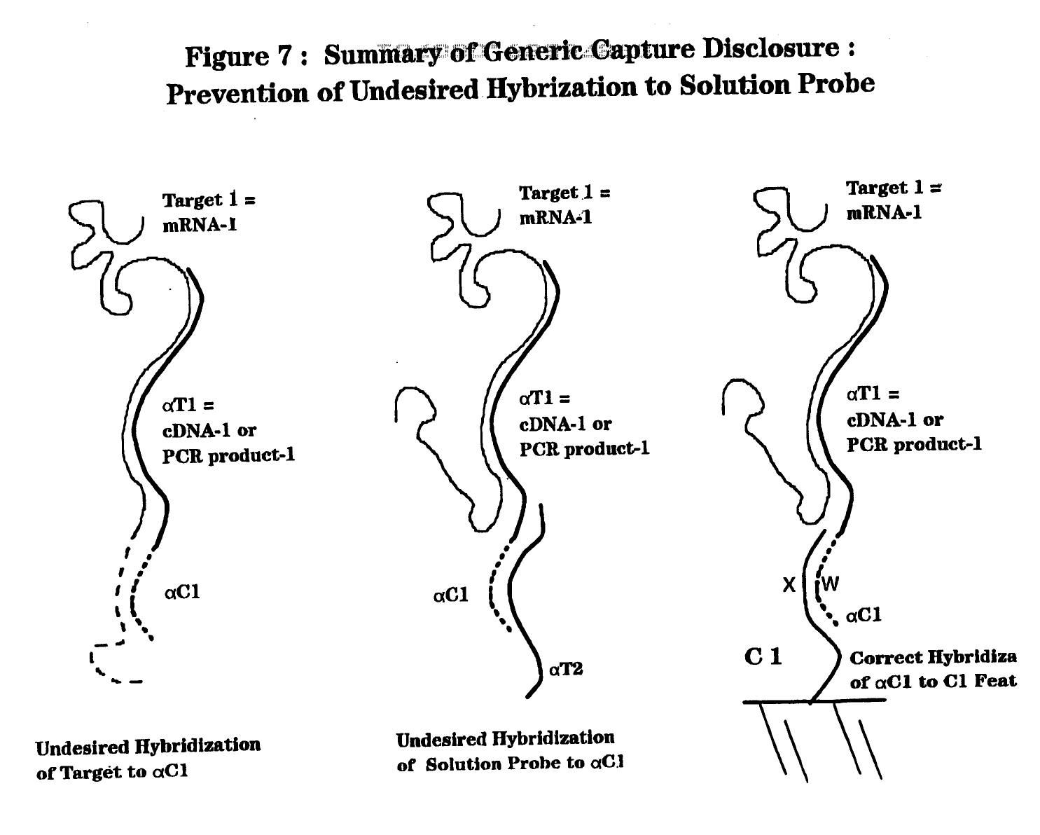

Increased specificity of solution probe hybridization to capture probes can be achieved by the use of modified or nonstandard nucleotides, such as isocytidine and isoguanosine, which do not base pair with standard nucleotides (FIG. 7; Scheit, Nucleotide Analogs (John Wiley, New York, 1980); Uhlman et al., Chemical Reviews 90:543-584, 1990). For example, use of pairs of such modified or nonstandard nucleotides in the αC region of a solution probe and the corresponding region of a capture probe can increase the likelihood of the occurrence of a specific interaction between these molecules, rather than, for example, undesired hybridization between the αC region of a solution probe and target molecules (FIG. 7). Such analogs can also be used to enhance binding properties and to reduce degeneracy, as is known by those of skill in the art.

Reverse polarity nucleotides can also be used in the probes of the invention, to achieve greater specificity between capture and solution probes (see, e.g., Koga et al., J. Org. Chem. 56(12):3757-3759, 1991; Koga et al., Nuc. Acids Symp. Series 29:3-4, 1993; Koga et al., J. Org. Chem. 60:1520-1530, 1995). Such nucleotides can be present throughout the region of basepairing, or interspersed in this region, as can be determined by one of skill in this art.

As is noted above, solution probes of the arrays of the invention can, optionally, be covalently linked to capture probes, so that they are stably bound to solid supports at temperatures above the melting temperatures of capture probe/αC complement duplexes consisting solely of standard nucleotides (i.e., deoxyadenosine, deoxythymidine, deoxycytidine, and deoxyguanine). This increased stability can be achieved by the use the following approaches, or combinations of these approaches.

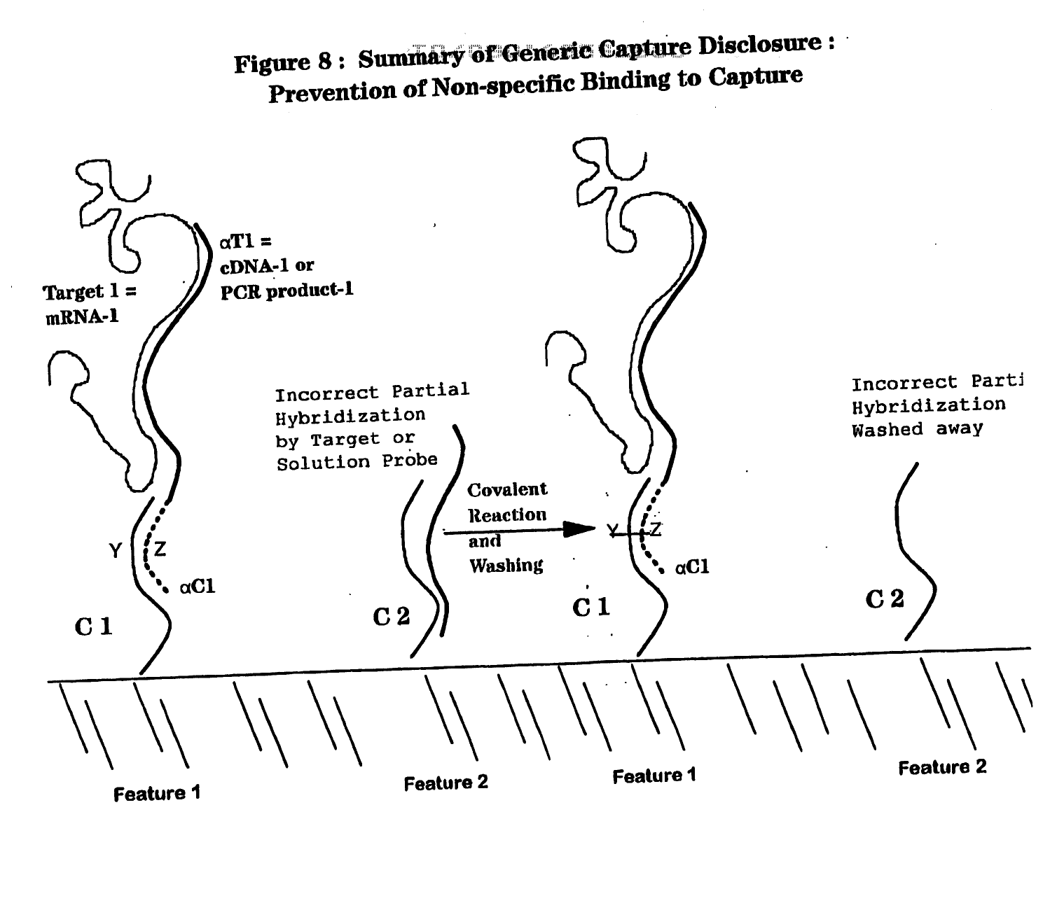

Increased stability of solution probe linkage to a substrate, via a capture probe, can be achieved by crosslinking the capture probe to the αC region of the solution probe (see, e.g., FIG. 8). Crosslinking of this duplex structure can be carried out using any of numerous methods known in the art. For example, a crosslinking agent, such as psoralen or ethidium bromide, which can be activated by exposure to light, can be applied to a hybrid formed between a capture probe and an αC region of a solution probe. Such intraduplex crosslinking can also be achieved by the incorporation of modified bases containing moieties that can be activated to covalently crosslink to one another in the hybridizing portions of the capture probe and solution probe αC region. Upon formation of crosslinks between such molecules, high stringency washing can be carried out, to wash away incorrect, partially hybridized molecules (FIG. 8). Examples of this type of cross-linking, in which aziridine is used to cross-link the modified bases, are given by Webb et al. (JACS 108:27645, 1986; Nucleic Acids Research 14:7661, 1986; Tetrahedron Letters 28:2469, 1987) and Cowart et al. (Biochemistry 30:788, 1991). Favre et al. (J. Photochemistry and Photobiology B-Biology 42:109-124, 1998) use thionucleobases, which when irradiated with UV light, initiate cross linking between the strands.

As is noted above, a central feature of the arrays of the invention is that at least some of the solution probes are directly linked to the solid support. This can be achieved, for example, by incorporating on one end of a solution probe a moiety that can covalently bind to the surface of a solid support upon activation. For example, a molecule containing an arylazide or fluorinated arylazide functionality can be incorporated at the end of a solution probe that is closest to a solid support upon hybridization of the solution probe to a capture probe. After hybridization at a low temperature, the arylazide can be activated by light, forming a highly active nitrene intermediate, which form a covalent bond with organic moieties present on the solid support. To increase the probability that the arylazide binds to the solid support, it can be attached to the end of a linker that interacts with the solid support by hydrophobic or charge interactions. Aryl azides covalently linked to a solid support have been used to create systems in which the surface bound nitrene reacts with molecules in solution (U.S. Pat. No. 4,562,157). Compounds other than arylazides are also useful for photoactivation. For example, peptides containing a benzophenone moiety, upon irradiation, have been shown to covalently bind to organic surfaces containing an active hydrogen (U.S. Pat. No. 4,762,881). Diazopyruvic acid amides undergo ultraviolet photolysis to form ketene amides. This reactive species will form covalent bonds with nucleophilic species such as amines or alcohols (Goodfellow et al., Biochemistry 28:6346-6360, 1989). Catalogs from Pierce (Rockford, Ill.) and Molecular Probes (Eugene, Oreg.) include a variety of bifunctional linkers that can be used to create suitable cross-linking between the solution probe and the surface.

Alternatively, a potentially active group, such as an amine or a thiol, can be present in a masked form. Deprotection of the group after hybridization, by either chemical or radiative means, frees it to react with the appropriate functionalities on the solid support, such as an isothiocyanate, activated ester, or maleimide functionality. Also, if desired, the capture oligonucleotide can be removed, by specific cleavage of a linker, after covalent binding between the solution probe and the solid support has been achieved. In any event, after covalent binding has taken place, as is discussed above, a high stringency wash can be carried out to wash away non-specifically bound molecules. This provides a significant advantage, because in methods that do not include such strategies, increasing the temperature of the system above the melting temperatures of any duplexes results in their melting, and the loss of any spatial information they provided to the array. In contrast, using the methods described above, the temperature of the system can be raised above the melting temperatures of any covalently linked duplexes, to eliminate non-specific binding.

Methods of Detecting Target Analytes in Samples

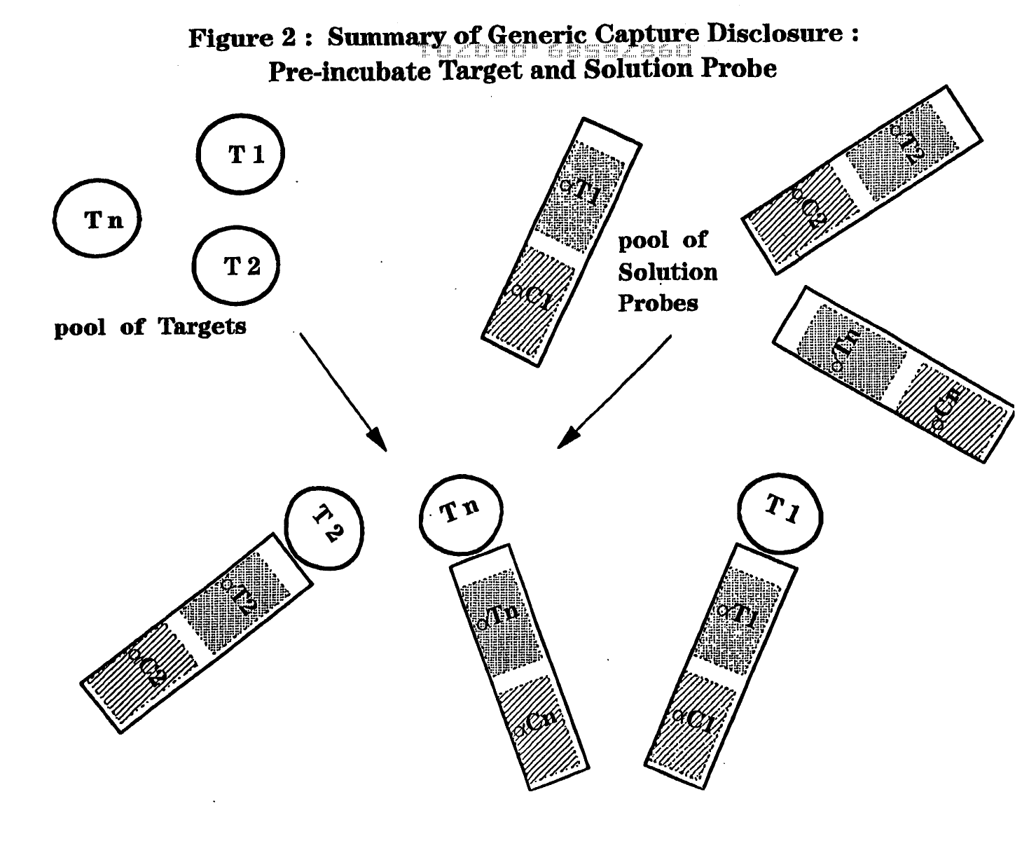

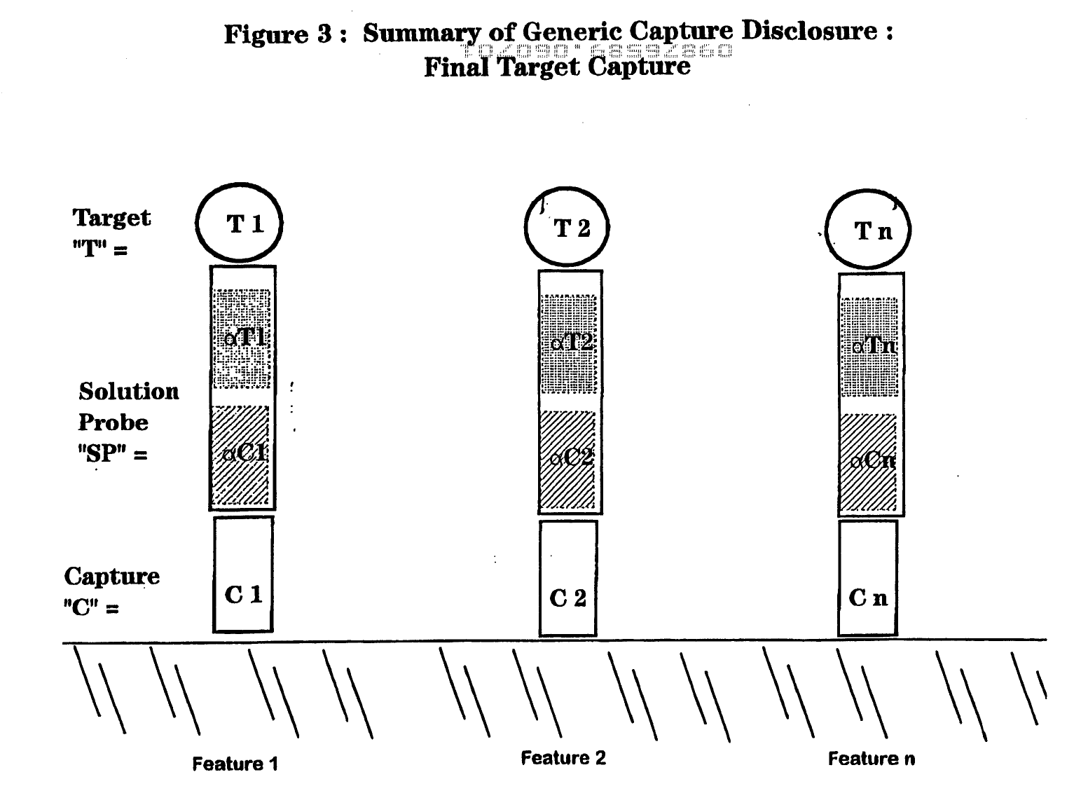

The capture probe arrays and solution probes described above can be used in numerous methods, for example, for detecting target nucleic acid molecules (RNA (e.g., mRNA or tRNA), DNA (e.g., genomic DNA or PCR products), or modifications thereof, that can be, e.g., derived from a pathogen or a host) in samples. For example, a target analyte and a solution probe can be pre-incubated with one another, in the absence of a capture probe array, to allow hybridization to occur in solution phase. Such pre-incubation can be carried out using a pool of many targets and a pool of many corresponding solution probes (FIG. 2). In this example, the concentration of solution probes can be equivalent to, or in excess of, the concentration of target analyte, e.g., a concentration that is as high as about 10 times the highest expected concentration of a corresponding target. After pre-incubation, the solution containing specifically bound target analyte and solution probes is applied to a capture probe array, and the capture probes, which are covalently bound to a solid support, now bind or hybridize to the αC regions of the solution probes (FIG. 3). The solution probes and capture probes are then covalently crosslinked or the solution probe is directly crosslinked to the solid support, as is described above. Detection of solution probes that have specifically bound to both target analyte and capture probes are then detected using standard methods.

Labels that can be used in the invention to facilitate detection include, for example, radiolabels, chromophores, fluorophores (e.g., fluorescein and rhodamine), chemiluminescent moieties, and transition metals. Methods for detecting such labels are well known in the art and include the use of, for example, labeled enzymes and labeled antibodies, as well as methods such as autoradiography, fluorescence microscopy, laser scanning, and the use of charge-coupled devices.

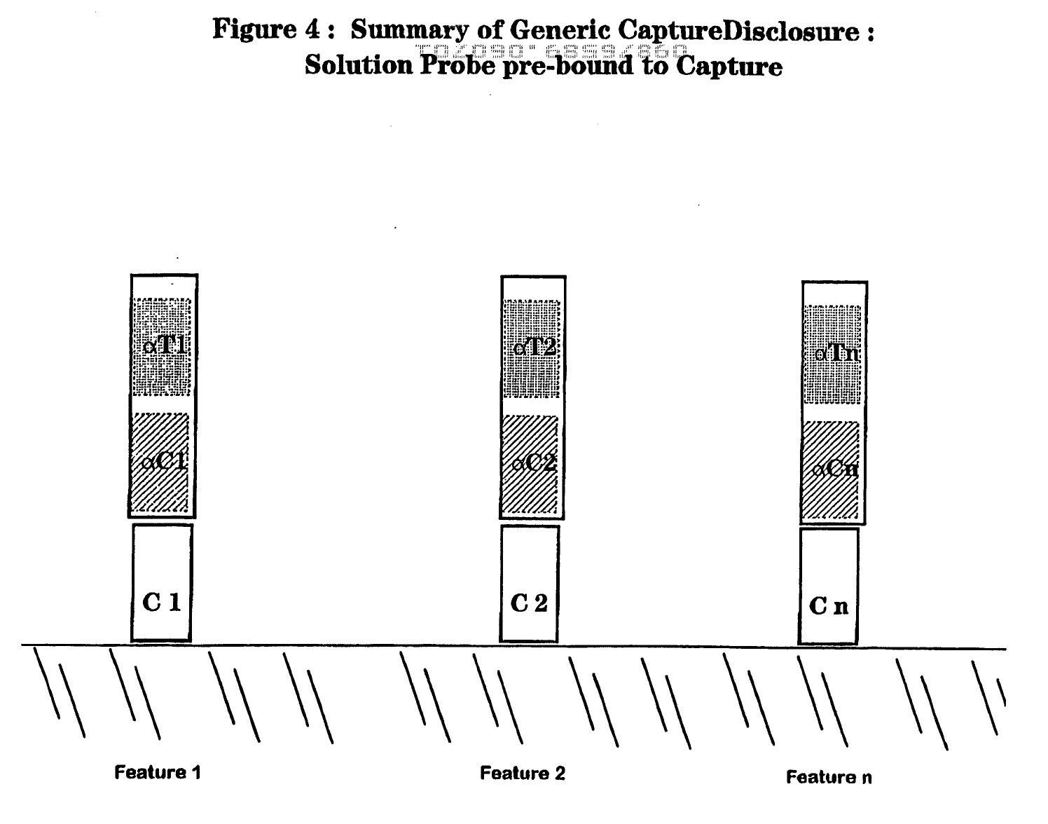

In a variation of the method described above, a competitive or sequential assay is carried out to quantitate the amount of a target analyte in a sample. In such an assay, a known amount of a labeled target analyte is pre-incubated with a pool of solution probes in the presence of a sample containing unlabeled target, and detection of the amount of labeled target bound to the capture array is used as a measure of the amount of unlabeled target present in the sample. In an alternative method of the invention, a different order of pre-incubation is used. That is, a pool of solution probes is pre-incubated with a capture probe array, in the absence of target analyte, which is added later (FIG. 4). Crosslinking of solution probes to capture probes or linking of solution probes to the solid support is carried out before addition of the target analyte.

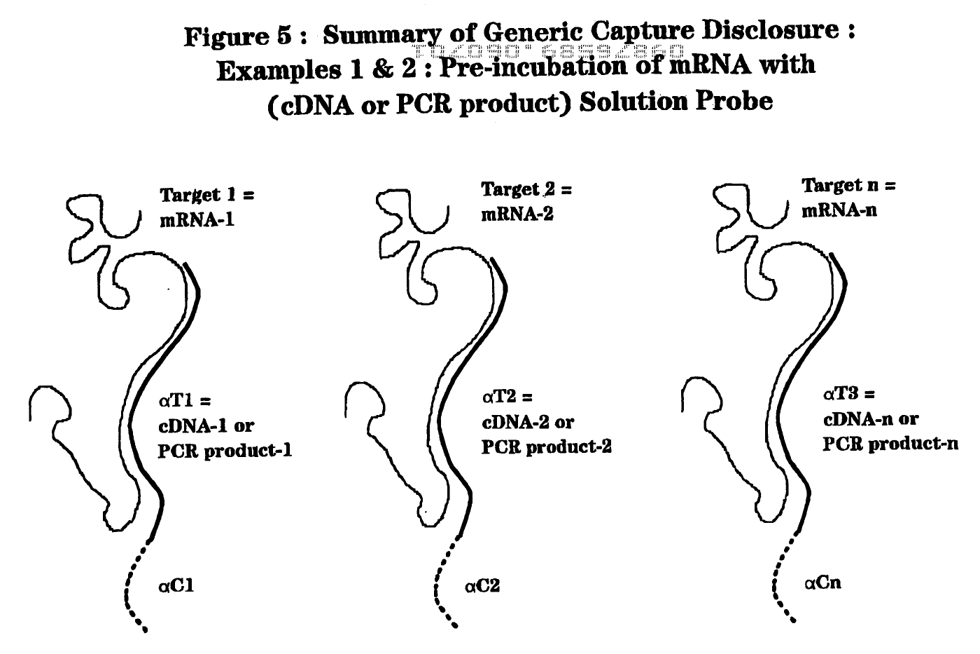

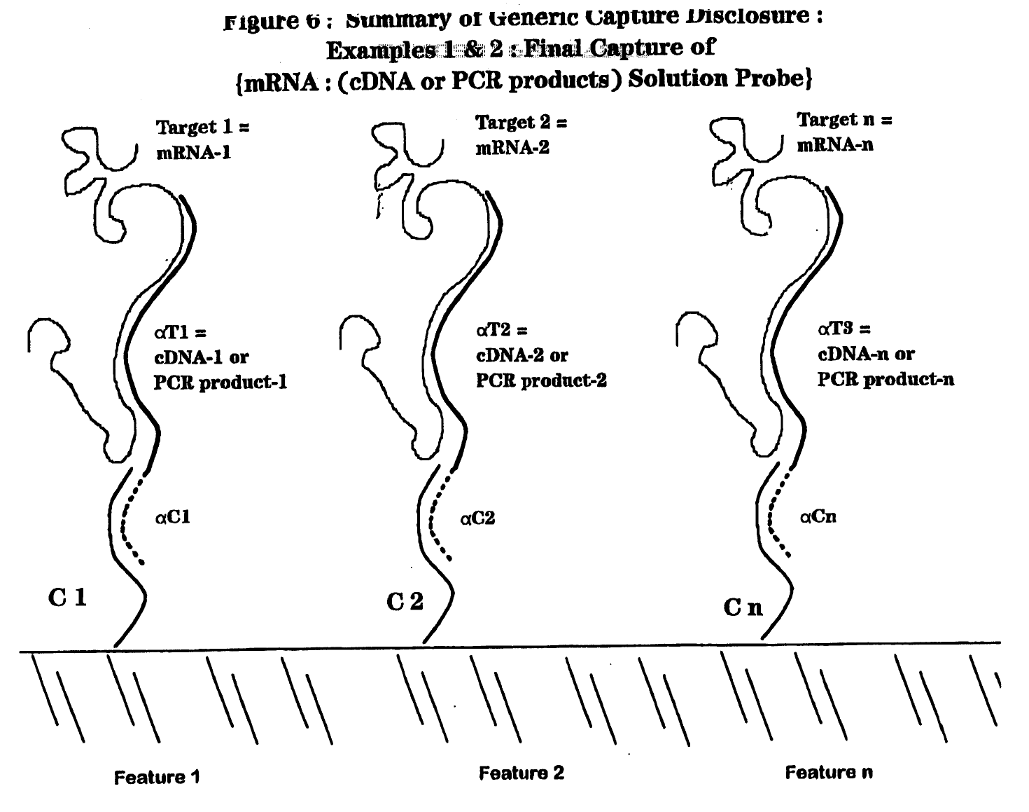

An additional method included in the invention is illustrated in FIG. 5. In this method, a cDNA or PCR product is used as the α-target region of a solution probe. The α-capture probe region of such a solution probe can be synthesized on the 5′ end of the primer used to generate the cDNA or PCR product, resulting in covalent linkage between the two portions of the solution probe. Hybridization of such a probe with target analytes can be carried out in solution, under high stringency conditions (e.g., at a high temperature), and then cooled for the surface capture phase (FIG. 6).

All publications cited herein are incorporated by reference in their entirety. Other embodiments are within the following claims.

What is claimed is:

1. An array of nucleic acid molecule probes linked to a solid support, said array comprising a plurality of (a) nucleic acid molecule capture probes bound to said solid support, and (b) nucleic acid molecule solution probes that each comprise (i) a first region that is specifically bound to a capture probe, and (ii) a second region that specifically binds to a target nucleic acid molecule analyte, wherein at least some of said solution probes are covalently linked to said solid support by a chemical moiety attached to said solution probes that, in an activated state, covalently binds to said support.

2. The array of claim 1, wherein interaction between at least some of said solution probes and at least some of said capture probes involves interaction between nonstandard or reverse polarity bases.

3. The array of claim 1, wherein at least some of said solution probes are covalently crosslinked to at least some of said capture probes.

4. The array of claim 1, comprising different solution probes that are specific for distinct target nucleic acid molecule analytes.

5. A method of making an array of nucleic acid molecule probes for detecting a target nucleic acid molecule analyte in a sample, said method comprising the steps of:
(a) providing an array comprising nucleic acid molecule capture probes bound to a solid support;
(b) contacting the array of step (a) with nucleic acid molecule solution probes that each comprise (i) a first region that specifically binds to a capture probe, and (ii) a second region that specifically binds to a target nucleic acid molecule analyte; and
(c) covalently linking at least some of said solution probes to said solid support by way of an activatable chemical moiety attached to said solution probes.

6. The method of claim 5, wherein interaction between at least some of said solution probes and at least some of said capture probes involves interaction between nonstandard or reverse polarity bases.

7. The method of claim 5, further comprising covalently crosslinking at least some of said solution probes to at least some of said capture probes.

8. The method of claim 5, wherein said array comprises capture probes that are specific for different solution probes, which are specific for distinct target nucleic acid molecule analytes.

9. A method of detecting a target nucleic acid molecule analyte in a sample, said method comprising the steps of:
(a) contacting a nucleic acid molecule capture probe, bound to a solid support, with a nucleic acid molecule solution probe comprising (i) a first region that specifically binds to a target analyte, and (ii) a second region that specifically binds to said capture probe;
(b) covalently linking said solution probe to said solid support;
(c) contacting the product of (b) with said sample; and
(d) monitoring said solid support for the presence of said target nucleic acid molecule analyte bound to said capture probe.

10. The method of claim 9, further comprising covalently crosslinking said solution probe to said capture probe.

11. The method of claim 9, further comprising detecting a second target nucleic acid molecule analyte in said sample.

12. The method of claim 9, further comprising detecting said target nucleic acid molecule analyte in more than one sample.

13. A method of detecting a target nucleic acid molecule analyte in a sample, said method comprising the steps of:
(a) contacting a nucleic acid molecule solution probe, comprising (i) a first region that specifically binds to a target nucleic acid molecule analyte, and (ii) a second region that specifically binds to a nucleic acid molecule capture probe, with said sample;
(b) contacting the product of (a) with said capture probe, bound to a solid support;
(c) covalently linking said solution probe to said solid support; and
(d) monitoring said solid support for the presence of said target nucleic acid molecule analyte bound to said capture probe.

14. The method of claim 13, further comprising covalently crosslinking said solution probe to said capture probe.

15. The method of claim 13, further comprising detecting a second target nucleic acid molecule analyte in said sample.

16. The method of claim 13, further comprising detecting said target nucleic acid molecule analyte in more than one sample.
